(19) United States
(12) Patent Application Publication
(10) Pub. No.: US 2024/0008765 A1
(43) Pub. Date: Jan. 11, 2024

(54) ESTABLISHING METHOD OF SLEEP APNEA ASSESSMENT PROGRAM, SLEEP APNEA ASSESSMENT SYSTEM, AND SLEEP APNEA ASSESSMENT METHOD

(71) Applicant: China Medical University, Taichung City (TW)

(72) Inventors: Kai-Cheng Hsu, Taichung City (TW); Liang-Wen Hang, Taichung City (TW); Ya-Lun Wu, Taichung City (TW); Meng-Hsuan Liu, Taichung City (TW)

(73) Assignee: China Medical University, Taichung City (TW)

(21) Appl. No.: 18/152,364

(22) Filed: Jan. 10, 2023

(30) Foreign Application Priority Data
Jul. 11, 2022 (TW) 111125970

(51) Int. Cl. A61B 5/08; A61B 5/364; A61B 5/00

(52) U.S. Cl. CPC A61B 5/0826; A61B 5/364; A61B 5/7264

(57) ABSTRACT

A sleep apnea assessment method includes the following steps. A sleep apnea assessment system is provided. A target ECG signal data of the subject is obtained. A data pre-processing step is performed so as to obtain a target ECG time-frequency data, and the target ECG time-frequency data is processed so as to obtain a plurality of target time-frequency segment data. An assessing step of apnea event is performed so as to output an assessing result of sleep apnea event of each of the plurality of target time-frequency segment data, and the assessing result of sleep apnea event is for assessing whether the subject has the sleep apnea event in any one of the plurality of target time-frequency segment data or not and predicting a probability of an occurrence of sleep apnea event of the subject.

### RELATED APPLICATIONS

This application claims priority to Taiwan Application Serial Number 111125970, filed Jul. 11, 2022, which is herein incorporated by reference.

### BACKGROUND

### Technical Field

The present disclosure relates to a medical information analysis method and a system thereof. More particularly, the present disclosure relates to an establishing method of sleep apnea assessment program, a sleep apnea assessment system and a sleep apnea assessment method.

### Description of Related Art

Sleep apnea is a common sleep disorder. Due to the problems of the incoordination between the muscles of the upper respiratory tract, the soft tissue in the pharynx being excessively bulky, the hyperplasia or the hypertrophy of the tonsils, etc., the respiratory tract of the patient suffering from sleep apnea will repeatedly collapse while sleeping. Thus, the breathing of the patient may stop or become shallow, and the symptoms such as insomnia, nocturia, disrupted sleep, abnormal snoring, and lower oxygen saturation in the blood may occur.

The diagnosis of sleep apnea is mainly based on the results obtained from the polysomnography (PSG) so as to assess whether the subject is a patient suffering from sleep apnea or not. However, during the polysomnography, the subject is required to stay overnight at the medical center and undergo long-term sleep monitoring for 6 to 8 hours. During the examination, patches with metal sensors should be attached to the body, a sensor should be placed on the nose and mouth, and an induction loop should be tied on the chest and abdomen so as to record the brain waves, eye waves, electrocardiogram, electrooculogram, oxygen saturation in blood, pulse, or other physiological information. Further, the chest and abdomen breathing movements and the sounds or images of breathing through the nose and mouth of the subject are recorded simultaneously. Then, the data collected from the aforementioned examinations are analyzed so as to assess whether the subject has a sleep apnea event or not. Thus, it is not only time-consuming but also leads to inaccurate assessments due to environmental changes or disturbances.

Therefore, how to rapidly and accurately assess that the sleep apnea occurs or not is a technical issue with clinical application values.

### SUMMARY

According to one aspect of the present disclosure, an establishing method of sleep apnea assessment program, which is for establishing a sleep apnea assessment program, includes the following steps. An ECG (electrocardiogram) signal database is obtained, wherein the ECG signal database includes a plurality of reference ECG signal data and a plurality of time-point data of apnea event of a plurality of patients suffering from sleep apnea, the plurality of reference ECG signal data are single lead electrocardiograms of the patients in a total sleep time thereof, and each of the plurality of reference ECG signal data corresponds to one of the plurality of time-point data of apnea event of one of the patients. A reference data pre-processing step is performed, wherein the plurality of reference ECG signal data are respectively processed with a reference signal transform processing so as to obtain a plurality of reference ECG time-frequency data, and each of the plurality of reference ECG time-frequency data is processed with a reference signal cutting processing so as to obtain a plurality of processed time-frequency segment data, wherein each of the plurality of reference ECG signal data corresponds to one of the plurality of reference ECG time-frequency data. A weighting step is performed, wherein the plurality of processed time-frequency segment data are respectively labeled based on the plurality of time-point data of apnea event, and the plurality of processed time-frequency segment data are processed with an additional weight setting so as to obtain a plurality of reference time-frequency segment data. A first training step is performed, wherein the plurality of reference time-frequency segment data are trained to achieve a convergence by a deep learning calculating module so as to obtain a neural network classifier, and then the plurality of reference time-frequency segment data of the patients are analyzed by the neural network classifier so as to output a plurality of training ECG features. A second training step is performed, wherein the plurality of training ECG features of the patients are averaged and then trained to achieve a convergence by a classifying algorithm module so as to obtain a machine algorithm classifier. The sleep apnea assessment program includes the neural network classifier and the machine algorithm classifier, and the sleep apnea assessment program is for assessing whether a subject has a sleep apnea event or not, predicting a probability of the sleep apnea event occurring in the subject, assessing a time point that the subject has the sleep apnea event, and assessing a sleep apnea condition of the subject.

According to another aspect of the present disclosure, a sleep apnea assessment system includes a processor and an ECG signal capturing device. The processor includes a data pre-processing module and the sleep apnea assessment program established by the establishing method of sleep apnea assessment program of the aforementioned aspect. The ECG signal capturing device is signally connected to the processor, wherein the ECG signal capturing device is for capturing a target ECG signal data of the subject, and the target ECG signal data is a single lead electrocardiogram of the subject in a total sleep time thereof.

According to further another aspect of the present disclosure, a sleep apnea assessment method includes the following steps. The sleep apnea assessment system of the aforementioned aspect is provided. The target ECG signal data of the subject is obtained, wherein the target ECG signal data is captured by the ECG signal capturing device and then transported to the sleep apnea assessment program. A data pre-processing step is performed, wherein the target ECG signal data is processed with a target signal transform processing by the data pre-processing module so as to obtain a target ECG time-frequency data, and the target ECG time-frequency data is processed with a target signal cutting processing by the data pre-processing module so as to obtain a plurality of target time-frequency segment data. An assessing step of apnea event is performed, wherein the plurality of target time-frequency segment data are respectively analyzed by the neural network classifier so as to output an assessing result of sleep apnea event of each of the plurality of target time-frequency segment data, and the assessing result of sleep apnea event is for assessing whether the subject has the sleep apnea event in any one of the plurality of target time-frequency segment data or not and predicting a probability of an occurrence of sleep apnea event of the subject.

### BRIEF DESCRIPTION OF THE DRAWINGS

The present disclosure can be more fully understood by reading the following detailed description of the embodiment, with reference made to the accompanying drawings as follows:

FIG. 1 is a flow chart of an establishing method of sleep apnea assessment program according to one embodiment of the present disclosure.

FIG. 2 is a block diagram of a sleep apnea assessment system according to another embodiment of the present disclosure.

FIG. 3 is a flow chart of a sleep apnea assessment method according to further another embodiment of the present disclosure.

FIG. 4 is a flow chart of a sleep apnea assessment method according to still another embodiment of the present disclosure.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

[Establishing Method of Sleep Apnea Assessment Program of the Present Disclosure]

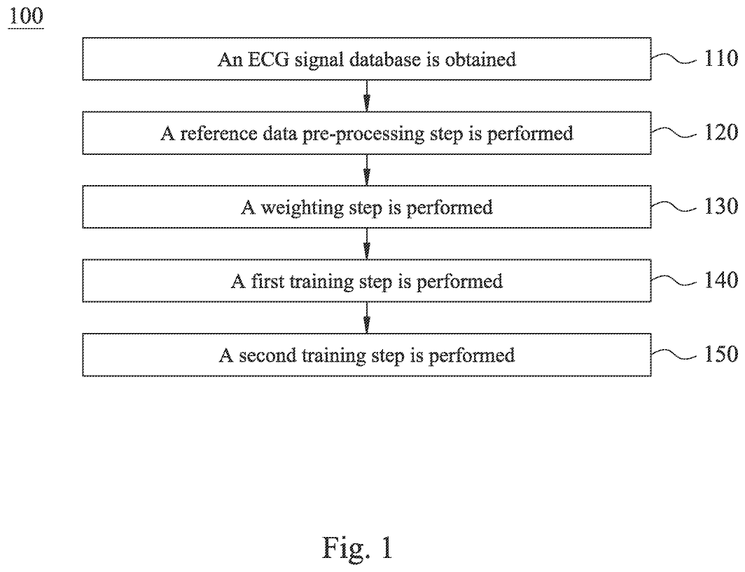

Reference is made to FIG. 1, which is a flow chart of an establishing method 100 of sleep apnea assessment program according to one embodiment of the present disclosure. The establishing method 100 of sleep apnea assessment program is for establishing a sleep apnea assessment program. The sleep apnea assessment program includes a neural network classifier and a machine algorithm classifier, and the establishing method 100 of sleep apnea assessment program includes Step 110, Step 120, Step 130, Step 140 and Step 150.

In Step 110, an ECG (electrocardiogram) signal database is obtained, wherein the ECG signal database includes a plurality of reference ECG signal data and a plurality of time-point data of apnea event of a plurality of patients suffering from sleep apnea. In detail, the reference ECG signal data are single lead electrocardiograms of the patients in a total sleep time thereof. The single lead electrocardiogram is more streamlined in information presentation and has high accuracy, so it is favorable for applying the sleep apnea assessment program of the present disclosure in wearable or portable mobile devices, and it has the advantages of simple operation and high accuracy.

Further, in the ECG signal database of the present disclosure, one patient suffering from sleep apnea corresponds to one of the reference ECG signal data in principle, or one patient suffering from sleep apnea can also correspond to more than one of the reference ECG signal data obtained at different time points, and the present disclosure is not limited thereto. However, each of the reference ECG signal data only corresponds to one of the time-point data of apnea event of one of the patients so as to facilitate the analysis of the time and the frequency of apnea events. The time-point data of apnea event includes the time points of apnea event and the times of apnea event, wherein the time points of apnea event are the start time and the end time of each of the apnea events of the patient suffering from sleep apnea in the total sleep time thereof. Furthermore, the ECG signal database can further include the information of gender, age, ethnicity, etc., of each of the patients suffering from sleep apnea so as to enhance the accuracy of the training, but the present disclosure is not limited thereto.

In Step 120, a reference data pre-processing step is performed, wherein the plurality of reference ECG signal data are respectively processed with a reference signal transform processing so as to obtain a plurality of reference ECG time-frequency data. Then, each of the plurality of reference ECG time-frequency data is processed with a reference signal cutting processing so as to obtain a plurality of processed time-frequency segment data. In detail, in the reference data pre-processing step, each of the reference ECG signal data will be processed with the reference signal transform processing so as to transform the reference ECG signal data to the reference ECG time-frequency data, and each of the reference ECG signal data corresponds to one of the reference ECG time-frequency data. The reference ECG time-frequency data is a two-dimensional color graph, wherein the horizontal axis thereof presents the time, the vertical axis thereof presents the frequency, and the brightness of the signal block represents the level of energy, wherein the higher the brightness is, the greater the energy is. Further, the reference signal transform processing can be Short-term Fourier transform (STFT). Compared to extracting some features of ECG (such as RR interval) for analysis, the reference ECG time-frequency data obtained from Short-term Fourier transform can more accurately reflect the characteristics of the apnea event.

Then, in the reference data pre-processing step, each of the reference ECG time-frequency data will be processed with a reference signal cutting processing so as to obtain a plurality of processed time-frequency segment data. In detail, after the reference ECG signal data are transformed to the reference ECG time-frequency data by the reference signal transform processing, each of the reference ECG time-frequency data will be processed with the reference signal cutting processing so as to divide each of the reference ECG time-frequency data into the plural of the processed time-frequency segment data for the following analysis. In particular, a length of each of the plurality of processed time-frequency segment data can be 60 seconds, and the reference signal cutting processing can be an overlapping signal cutting processing, so that there is a signal overlap region between every two of the plurality of processed time-frequency segment data adjacent to each other. In other words, each of the processed time-frequency segment data includes the information of the previous one and the next one of the said processed time-frequency segment data. Further, a length of the signal overlap region can be 30 seconds, but the present disclosure is not limited thereto. Furthermore, any signal processing module that can perform the reference signal transform processing and the reference signal cutting processing to the reference ECG signal data can be used to perform the reference data pre-processing step of the present disclosure, and the present disclosure is not limited to a specific type of signal processing module.

In Step 130, a weighting step is performed, wherein the plurality of processed time-frequency segment data are respectively labeled based on the plurality of time-point data of apnea event, and the plurality of processed time-frequency segment data are processed with an additional weight setting so as to obtain a plurality of reference time-frequency segment data. In detail, in the weighting step, all of the reference time-frequency segment data are labeled based on the time-point data of apnea event of the patients suffering from sleep apnea first so as to verify the occurrence of apnea event for each of the reference time-frequency segment data, and then the additional weight setting is performed to the reference time-frequency segment data that are labeled.

In particular, the additional weight setting will adjust the training weight of each of the reference time-frequency segment data according to the frequency of apnea events in the patients suffering from sleep apnea during the total sleep time. For example, in the normal condition without the apnea event, the weight of all of the reference time-frequency segment data is 1. However, when the ratio of the period with apnea event and the period without apnea event of the patient suffering from sleep apnea in the total sleep time thereof is 1:8, the weight of the reference time-frequency segment data with severe sleep apnea events is 8/9×2, and the weight of the reference time-frequency segment data without severe sleep apnea events is 1/9×2. Further, when the ratio of the period with apnea event and the period without apnea event of the patient suffering from sleep apnea in the total sleep time thereof is 2:5, the weight of the reference time-frequency segment data with severe sleep apnea events is 5/7×2, and the weight of the reference time-frequency segment data without severe sleep apnea events is 2/7×2, and so on. It must be noted that the aforementioned examples are only used to describe how to implement the methods of the present disclosure, and the present disclosure is not limited thereto.

Therefore, compared to the conventional weight setting method that uses all the data to calculate the weight ratio, the establishing method 100 of sleep apnea assessment program of the present disclosure independently calculates the reference ECG signal data of each of the sleep apnea patients suffering from sleep apnea. Thus, it is favorable for improving the efficiency for analyzing the data with extreme distribution, and the assessment accuracy of the sleep apnea assessment program established therefrom can be enhanced.

In Step 140, a first training step is performed, wherein the plurality of reference time-frequency segment data are trained to achieve a convergence by a deep learning calculating module so as to obtain a neural network classifier, and then the plurality of reference time-frequency segment data of the patients are analyzed by the neural network classifier so as to output a plurality of training ECG features. In particular, the deep learning calculating module can be EfficientNet deep learning calculating module. Further, although it is not shown in the figures, in the first training step, all of the reference time-frequency segment data after being processed by the weighting step can be divided into six groups based on the number of the patients suffering from sleep apnea, wherein five of the groups are used as the training sets and the validation set, and the other one is used as the test set so as to establish the neural network classifier of the present disclosure with a higher integrity, but the present disclosure is not limited thereto. Furthermore, after performing every of the first training step, the establishing method 100 of sleep apnea assessment program of the present disclosure can correct the parameters of the neural network classifier based on the training results obtained from the training of the neural network classifier as the feedback, so that the structure of the neural network classifier can be more in line with the actual situation and needs, but the present disclosure is not limited thereto.

In Step 150, a second training step is performed, wherein the plurality of training ECG features of the patients are averaged and then trained to achieve a convergence by a classifying algorithm module so as to obtain a machine algorithm classifier. In detail, after being processed by the aforementioned steps, the training ECG features can accurately reflect the occurrence of apnea events in different reference time-frequency segment data of the patient suffering from sleep apnea. Then, all of the training ECG features of each of the patients suffering from sleep apnea are averaged and then trained by the classifying algorithm module to achieve the convergence so as to obtain the machine algorithm classifier of the present disclosure. In particular, the classifying algorithm module can be Xgboost classifying algorithm module.

Therefore, the establishing method 100 of sleep apnea assessment program of the present disclosure uses numerous of the reference ECG signal data, and then the reference ECG signal data are analyzed and simulated with deep learning artificial intelligence models, accompanied by the transformation of the reference signal data, the sample segmenting with the overlap of the training time and additional weight setting, etc., so as to establish the sleep apnea assessment program. Hence, it is favorable for enhancing the feature intensity in time and space of the reference ECG signal data, and the ease of use and versatility in clinical applications of the sleep apnea assessment program established therefrom also can be effectively enhanced. Furthermore, the sleep apnea assessment program established by the establishing method 100 of sleep apnea assessment program of the present disclosure can be used to assess whether a subject has a sleep apnea event or not, predict a probability of the sleep apnea event occurring in the subject, assess a time point that the subject has the sleep apnea event, and assess a sleep apnea condition of the subject, and has excellent clinical application potential.

[Sleep Apnea Assessment System of the Present Disclosure]

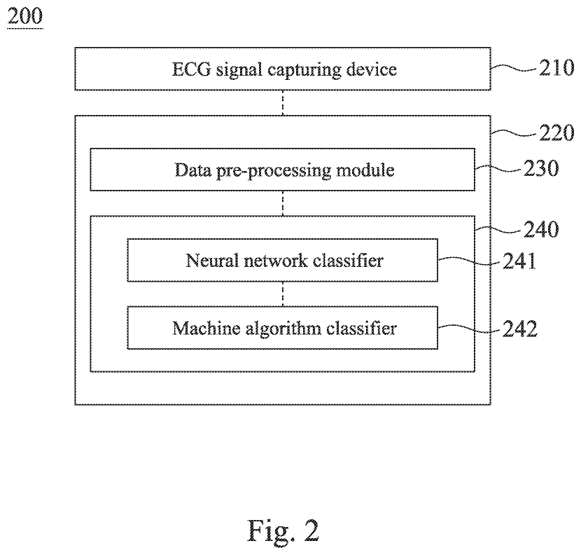

Reference is made to FIG. 2, which is a block diagram of a sleep apnea assessment system 200 according to another embodiment of the present disclosure. The sleep apnea assessment system 200 includes an ECG signal capturing device 210 and a processor 220.

The ECG signal capturing device 210 is for capturing a target ECG signal data of a subject. The target ECG signal data is a single lead electrocardiogram of the subject in a total sleep time thereof. Further, the ECG signal capturing device 210 can be the ECG signal capturing device used in the current clinical or the ECG signal capturing device built into wearable or portable mobile devices. In other words, any device that can capture the single lead electrocardiogram of the subject in the total sleep time thereof can be used as the ECG signal capturing device 210 of the present disclosure.

The processor 220 is signally connected to the ECG signal capturing device 210, and the processor 220 includes a data pre-processing module 230 and a sleep apnea assessment program 240. In particular, the sleep apnea assessment program 240 is established by the establishing method of sleep apnea assessment program of the present disclosure, and the sleep apnea assessment program 240 includes a neural network classifier 241 and a machine algorithm classifier 242. The ECG signal capturing device 210 can be connected to the processor 220 wirelessly or by wire, but the present disclosure is not limited thereto. Furthermore, any signal processing module that can perform data processing on ECG signal data can be used as the data pre-processing module 230 of the present disclosure, and the present disclosure is not limited to a specific type of signal processing module.

[Sleep Apnea Assessment Method of the Present Disclosure]

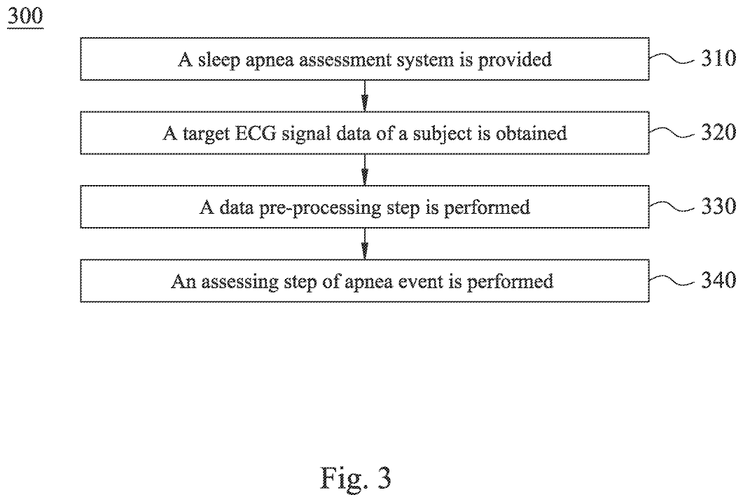

Reference is made to FIG. 2 and FIG. 3 simultaneously, and FIG. 3 is a flow chart of a sleep apnea assessment method 300 according to further another embodiment of the present disclosure. The sleep apnea assessment method 300 includes Step 310, Step 320, Step 330 and Step 340, and the steps and the details of the sleep apnea assessment method 300 of the present disclosure will be further illustrated in accompanying with the sleep apnea assessment system 200 of FIG. 2.

In Step 310, the sleep apnea assessment system 200 is provided.

In Step 320, a target ECG signal data of a subject is obtained, wherein the target ECG signal data is captured by the ECG signal capturing device 210 and then transported to the sleep apnea assessment program 240 for following analysis.

In Step 330, a data pre-processing step is performed, wherein the target ECG signal data is processed with a target signal transform processing by the data pre-processing module 230 so as to obtain a target ECG time-frequency data, and the target ECG time-frequency data is processed with a target signal cutting processing by the data pre-processing module so as to obtain a plurality of target time-frequency segment data. In detail, in the data pre-processing step, the target ECG signal data will be processed with the target signal transform processing so as to transform the target ECG signal data to the target ECG time-frequency data. The target ECG time-frequency data is a two-dimensional color graph wherein the horizontal axis thereof presents the time, the vertical axis thereof presents the frequency, and the brightness of the signal block represents the level of energy, wherein the higher the brightness is, the greater the energy is. Then, the target ECG time-frequency data will be processed with a target signal cutting processing so as to obtain a plurality of target time-frequency segment data. The target signal transform processing can be Short-term Fourier transform, and a length of each of the plurality of target time-frequency segment data can be 60 seconds. The target signal cutting processing can be an overlapping signal cutting processing, so that there is a signal overlap region between every two of the plurality of target time-frequency segment data adjacent to each other. In other words, each of the target time-frequency segment data includes the information of the previous one and the next one of the said target time-frequency segment data, and a length of the signal overlap region can be 30 seconds, but the present disclosure is not limited thereto. It must be noted that, the length of each of the target time-frequency segment data should be the same as the length of each of the processed time-frequency segment data, and the length of the signal overlap region between every two of the plurality of target time-frequency segment data adjacent to each other should be the same as the length of the signal overlap region between every two of the plurality of processed time-frequency segment data adjacent to each other. Thus, the demand for accurate assessment can be achieved.

In Step 340, an assessing step of apnea event is performed, wherein the plurality of target time-frequency segment data are respectively analyzed by the neural network classifier 241 so as to output an assessing result of sleep apnea event of each of the plurality of target time-frequency segment data, and the assessing result of sleep apnea event is for assessing whether the subject has the sleep apnea event in any one of the plurality of target time-frequency segment data or not and predicting a probability of an occurrence of sleep apnea event of the subject.

Therefore, by analyzing the target ECG signal data of the subject on the total sleep time thereof, the sleep apnea assessment method 300 of the present disclosure can effectively predict the probability of the sleep apnea event occurring in the subject at different time points of the total sleep time thereof and the time when the sleep apnea event occurred in the subject based on the information included in the target ECG signal data. Hence, it is favorable for assessing whether the subject is a patient suffering from sleep apnea and then planning for follow-up medical treatment in advance, so that the probability of cardiovascular diseases and chronic diseases caused by apnea events during sleep can be reduced.

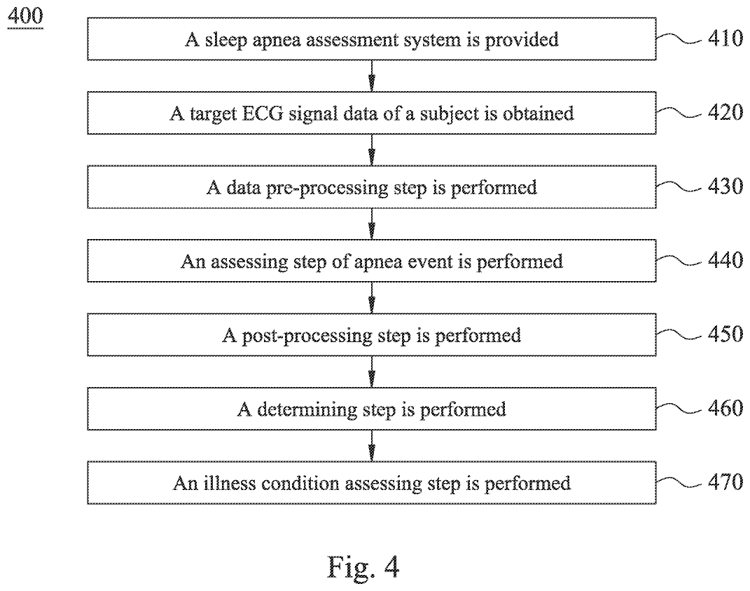

Reference is further made by FIG. 2 and FIG. 4 simultaneously, and FIG. 4 is a flow chart of a sleep apnea assessment method 400 according to still another embodiment of the present disclosure. The sleep apnea assessment method 400 includes Step 410, Step 420, Step 430, Step 440, Step 450, Step 460 and Step 470, wherein Step 410, Step 420, Step 430 and Step 440 are the same as Step 310, Step 320, Step 330 and Step 340 of FIG. 3, so that the same details there between are not described again herein. In particular, when the subject is determined as having the sleep apnea event by the assessing step of apnea event, the sleep apnea assessment method 400 will further compare and correct the target time-frequency segment data of the subject so as to assess the illness condition of sleep apnea of the subject. The steps and the details of the sleep apnea assessment method 400 of the present disclosure will be further illustrated in accompanying with the sleep apnea assessment system 200 of FIG. 2.

In Step 450, a post-processing step is performed, wherein the plurality of target time-frequency segment data are compared and corrected by the data pre-processing module 230 based on an occurring probability of apnea event of the assessing result of sleep apnea event of each of the plurality of target time-frequency segment data so as to obtain a plurality of processed target time-frequency segment data. In detail, the assessing result of sleep apnea event that is trained and obtained from the neural network classifier can include the occurring probability of apnea event of the subject, and whether the subject is the patient suffering from sleep apnea can be assessed therefrom. When the subject is determined as having the sleep apnea event by the sleep apnea assessment program 240 of the present disclosure, in the post-processing step, each of the target time-frequency segment data will be further corrected based on the occurring probability of apnea event so as to output the processed target time-frequency segment data for the following analysis.

In Step 460, a determining step is performed, wherein the plurality of processed target time-frequency segment data are analyzed by the neural network classifier so as to obtain a plurality of target ECG features.

In Step 470, an illness condition assessing step is performed, wherein the target ECG features are averaged and then analyzed by the machine algorithm classifier so as to output an assessing result of sleep apnea condition, and the assessing result of sleep apnea condition is for assessing the subject is a patient with mild sleep apnea, a patient with moderate sleep apnea or a patient with severe sleep apnea. In particular, because the clinical cases of the patients having mild sleep apnea or moderate sleep apnea are fewer. Thus, by the processing with the post-processing step, the determining step and the illness condition assessing step of the sleep apnea assessment method 400 of the present disclosure, it is favorable for effectively and accurately assessing the illness condition of the subject suffering from sleep apnea. Thus, it is favorable for planning the following medical treatments in advance and preventing the patient's health from being affected by the deterioration of the illness condition.

### Example

I. ECG Signal Database

The ECG signal database used in the present disclosure includes the single lead electrocardiograms of the patients suffering from sleep apnea in the total sleep time thereof collected by China Medical University Hospital. The ECG signal database includes the single lead electrocardiograms of different subjects and the time-point data of apnea event of each of the subjects during the total sleep time thereof.

The following experiments are performed by the sleep apnea assessment system of the present disclosure with the sleep apnea assessment method of the present disclosure so as to assess the accuracy for assessing the sleep apnea and the accuracy for assessing the illness condition of the apnea assessment system and the sleep apnea assessment method of the present disclosure. The sleep apnea assessment system of the present disclosure can be the sleep apnea assessment system 200, the sleep apnea assessment program 240 of the sleep apnea assessment system 200 can be established by the establishing method 100 of sleep apnea assessment program, and the sleep apnea assessment method of the present disclosure can be the sleep apnea assessment method 300 or the sleep apnea assessment method 400, so that the same details will not be described again herein.

II. Analyzing the Credibility of the Sleep Apnea Assessment System and the Sleep Apnea Assessment Method of the Present Disclosure

Reference is made to Table 1, which shows the assessing results of the sleep apnea assessment system and the sleep apnea assessment method of the present disclosure used to assess the sleep apnea condition of the subjects. In particular, when the subject is determined as the patient suffering from sleep apnea by the sleep apnea assessment system of the present disclosure, the target time-frequency segment data of the said subject will be corrected by the sleep apnea assessment program. Then, an assessing result of sleep apnea condition will be output by the sleep apnea assessment program, wherein the subject will further be assessed as a patient with mild sleep apnea, a patient with moderate sleep apnea or a patient with severe sleep apnea.

TABLE 1

| | Accuracy | Sensitivity | Specificity | AUROC |
|---|---|---|---|---|
| Mild | 0.812 | 0.804 | 0.924 | 0.901 |
| Moderate | 0.909 | 0.862 | 0.934 | 0.961 |
| Severe | 0.928 | 0.857 | 0.945 | 0.975 |

As shown in Table 1, all of the accuracy, the sensitively and the specificity of the sleep apnea assessment system and the sleep apnea assessment method of the present disclosure used to assess that the subject is a patient with mild sleep apnea, a patient with moderate sleep apnea or a patient with severe sleep apnea are excellent, and the AUROCs (Area Under the Receiver Operating Characteristic curve) thereof are up to 0.90 or more. Therefore, the sleep apnea assessment system and the sleep apnea assessment method of the present disclosure can be used to assess whether a subject has a sleep apnea event or not, predict a probability of the sleep apnea event occurring in the subject, assess a time point that the subject has the sleep apnea event, and assess a sleep apnea condition of the subject, and has excellent clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. An establishing method of sleep apnea assessment program, which is for establishing a sleep apnea assessment program, comprising:
obtaining an ECG (electrocardiogram) signal database, wherein the ECG signal database comprises a plurality of reference ECG signal data and a plurality of time-point data of apnea event of a plurality of patients suffering from sleep apnea, the plurality of reference ECG signal data are single lead electrocardiograms of the patients in a total sleep time thereof, and each of the plurality of reference ECG signal data corresponds to one of the plurality of time-point data of apnea event of one of the patients;
performing a reference data pre-processing step, wherein the plurality of reference ECG signal data are respectively processed with a reference signal transform processing so as to obtain a plurality of reference ECG time-frequency data, and each of the plurality of reference ECG time-frequency data is processed with a reference signal cutting processing so as to obtain a plurality of processed time-frequency segment data, wherein each of the plurality of reference ECG signal data corresponds to one of the plurality of reference ECG time-frequency data;
performing a weighting step, wherein the plurality of processed time-frequency segment data are respectively labeled based on the plurality of time-point data of apnea event, and the plurality of processed time-frequency segment data are processed with an additional weight setting so as to obtain a plurality of reference time-frequency segment data;
performing a first training step, wherein the plurality of reference time-frequency segment data are trained to achieve a convergence by a deep learning calculating module so as to obtain a neural network classifier, and then the plurality of reference time-frequency segment data of the patients are analyzed by the neural network classifier so as to output a plurality of training ECG features; and
performing a second training step, wherein the plurality of training ECG features of the patients are averaged and then trained to achieve a convergence by a classifying algorithm module so as to obtain a machine algorithm classifier;
wherein the sleep apnea assessment program comprises the neural network classifier and the machine algorithm classifier, and the sleep apnea assessment program is for assessing whether a subject has a sleep apnea event or not, predicting a probability of the sleep apnea event occurring in the subject, assessing a time point that the subject has the sleep apnea event, and assessing a sleep apnea condition of the subject.

2. The establishing method of claim 1, wherein the reference signal transform processing is Short-term Fourier transform (STFT).

3. The establishing method of claim 1, wherein a length of each of the plurality of processed time-frequency segment data is 60 seconds.

4. The establishing method of claim 1, wherein a signal overlap region is between every two of the plurality of processed time-frequency segment data adjacent to each other.

5. The establishing method of claim 4, wherein a length of the signal overlap region is 30 seconds.

6. The establishing method of claim 1, wherein the deep learning calculating module is EfficientNet deep learning calculating module.

7. The establishing method of claim 1, wherein the classifying algorithm module is Xgboost classifying algorithm module.

8. A sleep apnea assessment system, comprising:
a processor comprising a data pre-processing module and the sleep apnea assessment program established by the establishing method of sleep apnea assessment program of claim 1; and
an ECG signal capturing device signally connected to the processor, wherein the ECG signal capturing device is for capturing a target ECG signal data of the subject, and the target ECG signal data is a single lead electrocardiogram of the subject in a total sleep time thereof.

9. A sleep apnea assessment method, comprising:
providing the sleep apnea assessment system of claim 8;
obtaining the target ECG signal data of the subject, wherein the target ECG signal data is captured by the ECG signal capturing device and then transported to the sleep apnea assessment program;
performing a data pre-processing step, wherein the target ECG signal data is processed with a target signal transform processing by the data pre-processing module so as to obtain a target ECG time-frequency data, and the target ECG time-frequency data is processed with a target signal cutting processing by the data pre-processing module so as to obtain a plurality of target time-frequency segment data; and
performing an assessing step of apnea event, wherein the plurality of target time-frequency segment data are respectively analyzed by the neural network classifier so as to output an assessing result of sleep apnea event of each of the plurality of target time-frequency segment data, and the assessing result of sleep apnea event is for assessing whether the subject has the sleep apnea event in any one of the plurality of target time-frequency segment data or not and predicting a probability of an occurrence of sleep apnea event of the subject.

10. The sleep apnea assessment method of claim 9, wherein the target signal transform processing is Short-term Fourier transform.

11. The sleep apnea assessment method of claim 9, wherein a length of each of the plurality of target time-frequency segment data is 60 seconds.

12. The sleep apnea assessment method of claim 9, wherein a signal overlap region is between every two of the plurality of target time-frequency segment data adjacent to each other.

13. The sleep apnea assessment method of claim 12, wherein a length of the signal overlap region is 30 seconds.

14. The sleep apnea assessment method of claim 9, wherein when the subject has the sleep apnea event, the sleep apnea assessment method further comprises:
performing a post-processing step, wherein the plurality of target time-frequency segment data are compared and corrected by the data pre-processing module based on an occurring probability of apnea event of the assessing result of sleep apnea event of each of the plurality of target time-frequency segment data so as to obtain a plurality of processed target time-frequency segment data;
performing a determining step, wherein the plurality of processed target time-frequency segment data are analyzed by the neural network classifier so as to obtain a plurality of target ECG features; and
performing an illness condition assessing step, wherein the target ECG features are averaged and then analyzed by the machine algorithm classifier so as to output an assessing result of sleep apnea condition, and the assessing result of sleep apnea condition is for assessing the subject is a patient with mild sleep apnea, a patient with moderate sleep apnea or a patient with severe sleep apnea.
